(19) United States
(12) Patent Application Publication
(10) Pub. No.: US 2008/0030303 A1
(43) Pub. Date: Feb. 7, 2008

(54) Surgical pad accounting system and method

(76) Inventors: Myles Kobren, (US); Andrew Menzin, (US)

Correspondence Address: ELIOT GERBER, 1217 SKYCREST DR. #4, WALNUT CREEK, CA, 94595, US

(21) Appl. No.: 11/499,605

(22) Filed: Aug. 4, 2006

(51) Int. Cl. A61F 13/15

(52) U.S. Cl. 340/5.92; 604/362; 340/568.1

(57) ABSTRACT

In a surgical procedure absorbent pads, such as sterile gauze lap pads and sponges are used to absorb blood and other fluids. It is vital that none of the pads be left in the patient after the operation.

To account for each pad and be certain none are left in the patient, each pad is marked with unique human readable alphanumeric character, for example each pad in its face is printed with one number from the numbers 1,2,3 . . . 10 or more.

Similarly a container has pockets, each pocket being sealed on its top and two sides and with its top open. The surface of each pocket is marked with a alphanumeric human readable character, matching the characters on the pads. During ,or after the operation, each used pad is placed in its matching pocket i.e. pad “6” is placed in pocket “6”. A visual inspection reveals if any used pad is not in its matching packet which may indicate the pad has been incorrectly retained within the patient's body.

### BACKGROUND OF THE INVENTION

1. Technical Field

The invention relates generally to an apparatus and method for counting surgical supplies, and more specifically, to counting and accounting for all disposable surgical pads (lap pads or sponges) used in a surgical operation.

2. Related Art

During surgical procedures, absorbent pads, lap pads or sponges are employed to soak up blood and other fluids in and around the incision site. A lap pad or sponge left inside a patient is presumed to indicate that negligent care has taken place. Clearly, it is in both in a patient's and the health care provider's best interest to account for every surgical lap pad and sponge used in any particular surgical procedure.

The importance of accounting for all surgical pads, and not leaving any in the patient, is explained in “The Retained Surgical Sponge” by Drs. Verna Gibbs and Andrew Auerbach, in Ch. 22 of “Making Health Care Safer, A Critical Analysis of Patient Safety Practices” (Dept. of Health and Human Services, Evidence Report/Technology Assessment No. 43-AHRQ Pub. No. 01-E058, July 2001).

The term “surgical pad” is used herein to include absorbent pads including lap pads, laparotomy sponges and small sterile gauge pads (sponges). Typically a lap pad is 4 or 18 inches by 18 inches and a sponge is much smaller.

Surgical pads used in all surgical operations are typically packaged in containers having 5 to 10 pads therein. As they are used in the operating room, they are collected for later counting. An accurate counting of the number of pads is critical to assure that nothing unwanted is left behind in a incision.

The problem of retained pads is significant and not uncommon. It leads to significant patient illness and possible mortality. It is major cause of negligence cases. It is standard practice to obtain an X-ray of the operative site when the “counts” that are taken of all the pads and instruments are incorrect. Due to radio-opaque markers incorporate into the pads or sponges, the pads and instruments are detectable on a standard X-ray. However when the counts are correct, there may be retained items due to human error in counting. This is the heart of this persistent problem.

During surgery, when accounting for used sponges, the nurses or others assisting in the operation, count the used pads as time allows. They are then collected in many ways, such as in plastic bags, in bundles of five or ten sponges tied together, or in special devices used for counting the sponges. Many different types of special sponge/lap pad counting containers have been developed, the following U.S. patents merely being examples: U.S. Pat. Nos. 3,613,899; 3,749,237; 3,948,390; 4,190,153; 4,312,447; 4,372,102; 4,422,548; 4,429,789; 4,784,267; and 4,832,198.

Generally when a surgical procedure is completed, the doctor should request that the total number of sponges used should be accounted for. The nurses check their counts to see how many sponges have been used and whether the number used equals the number actually employed during the operation. If so, the incision is closed and the operation is completed. However, the original count may have been incorrect.

If not all sponges have been accounted for, a recount should be requested and all sponges should be counted one-by-one before the incision is closed. When using one of the prior art sponge counters identified above, this procedure may be accelerated, but the sponge counters may be complicated and expensive devices.

Sometimes sponge counts are performed multiple times during a surgical procedure, both at the beginning and throughout the procedure as pads are added, before closure of a deep incision or body cavity, and during personnel breaks and shift changes. Thus, within all the activity of an operating room, maintaining an accurate pad count is difficult, even if the original count is correct.

The patent literature suggests various products to make the procedure both simpler and more reliable. For example, various systems facilitate the hand-counting of surgical sponges by arranging the sponges into groups of arrangements to be visually inspected (see U.S. Pat. Nos. 4,364,490; 4,925,048 and 5,658,077). These systems may be problematic because medical personnel may determine blood loss by means of visual inspection or a manual weighing of soiled sponges and so soiled sponges are typically kept in one area of an operating room during a surgical procedure, thus creating the possibility that groupings are co-mingled or counted twice. In addition, operating room workers are often too rushed, fatigued and/or distracted to accurately count a large number of soiled sponges lumped together in one or more groups. This method also depends upon the accuracy of an initial count and, if the number of sponges in the original package is mislabeled by the manufacturer, then a missing sponge may be missed during a final count.

A second solution to the surgical sponge tracking problem is the a radio-opaque thread within the sponges. A radiopaque thread can be identified and located if a sponge is accidentally left inside a patient. A sponge that has been left in the body can be detected by X-ray. Various companies market sponges with radio-opaque threads. This solution is useful only if every patient is X-rayed after every operation.

A third solution to the pad/count problem is the inclusion of a radio frequency identification (RFID) tag in each pad (see U.S. Pat. Nos. 5,923,001; 5,931,824 and Application 20060044137). The RFID tag enables a patient to be scanned to detect the presence of a pad within a body cavity, but RFID tags may cost several times what a typical surgical pad costs and may be bulky, impairing the usefulness of the pad.

A fourth solution to the pad retention problem is a device that counts sponges as they are dropped, one-by-one, into an opening, or “entry gate” of the device (see U.S. Pat. No. 5,629,498). This solution is restricted by the accuracy of the original count and the precision of operating room assistants as they separate sponges from one another and drop them into the entry gate.

A fifth solution involves attaching a radio-opaque magnetic resonance device, or marker tag, to each pad which is them scanned by appropriate equipment (see U.S. Pat. Nos. 5,057,095; 5,644,582; 6,026,818 and 7,011,366). A problem with this solution is that marker tags and the scanning equipment are expensive and do not necessarily work well in an operating room environment.

### BRIEF SUMMARY OF THE INVENTION

The present invention provides a system and method to improve the safety of patients during surgical operations. Each of the surgical pads (lap pads, sponges, and other absorbent sterile pads) is accounted for before the surgical incision in the patient's body is closed.

Each surgical pad is printed, or otherwise marked, with a human readable alphanumeric character, for example the numbers 1,2,3 . . . 10 or more, each character being 2 to 6 inches high. Each surgical pad is marked with a different character, except if colors are used, which they may be. If different colors are used, each color has its own set of different characters. Also the pads preferably contain radio-opaque components.

A container for used pads has pockets which are printed, or otherwise marked, with matching numbers.

The surgical pads are preferably supplied in sterile bundles. Alternatively, the pads may be supplied in the marked pockets. In both cases, there is a one-to-one relationship between the marking i.e. numbers, on the pockets and the markings on the pad, both sets of markings being human readable alphanumeric characters.

During, or at the end of a surgical operation, each used pad is inserted, for example by a nurse or other assistant, into its matching pocket; for example, lap pad “6” is inserted into pocket “6”. In this way, a visual inspection of the container will readily and assuredly ascertain if any pads are missing i.e. if any pockets are empty the pad may be retained in the patient.

### BRIEF DESCRIPTION OF SEVERAL VIEWS OF THE DRAWING

In the accompanying drawings; and in accordance with embodiments of the present invention;

FIG. 1A is a top plan view of the front face of a surgical lap pad;

FIG. 1B is a top plan view of the back face of a surgical lap pad:

FIG. 2 is a top plan view of a surgical sponge;

FIGS. 3A and 3B are top plan views of two preferred embodiments of parts of containers for a set of lap pads of FIG. 1, the containers being empty; and

FIG. 4 is similar to FIG. 3 but with the container filled with lap pads.

### DETAILED DESCRIPTION OF THE INVENTION

The present invention relates to improving the safety of patients during surgical operations. The term, as used herein, of “surgical pads” refers to absorbent pads used in a surgical procedure to absorb blood or other fluids. One type of surgical pad is a sterile gauze pad called a “lap pad” or “laparodermy pad” which typically is 4 or 16 inches long by 16 inches wide. Another type of surgical pad is a surgical sponge which typically is of sterile gauze and may be 2 inches square in size.

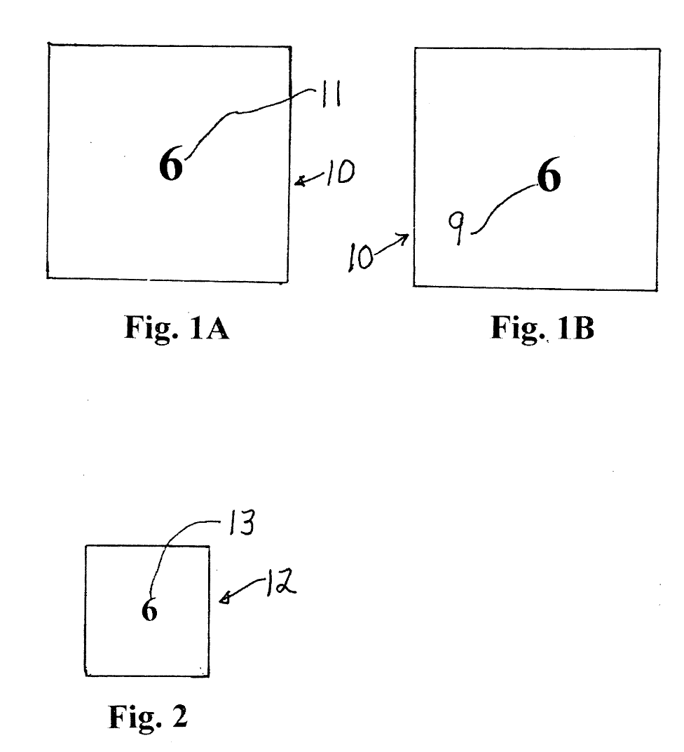

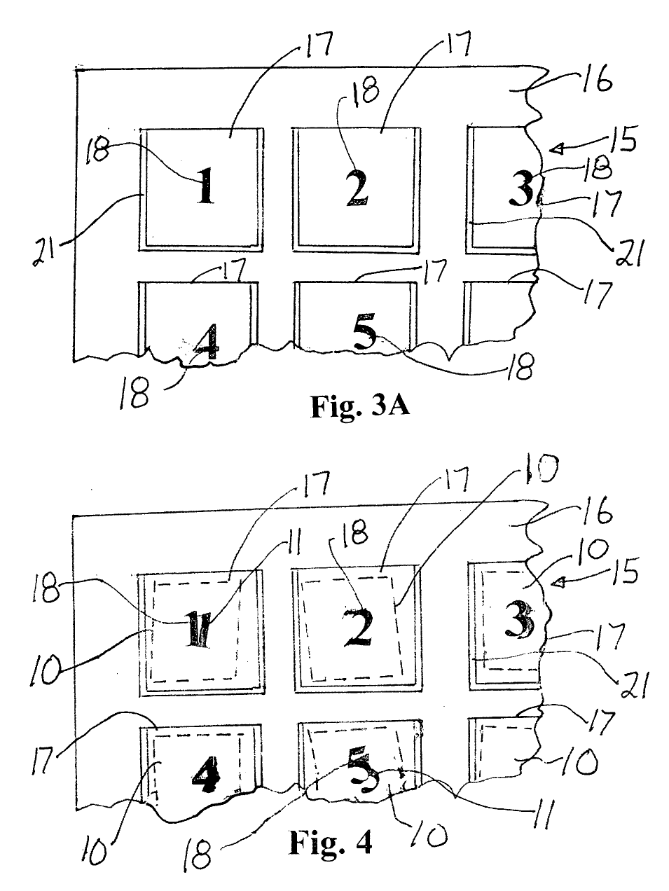

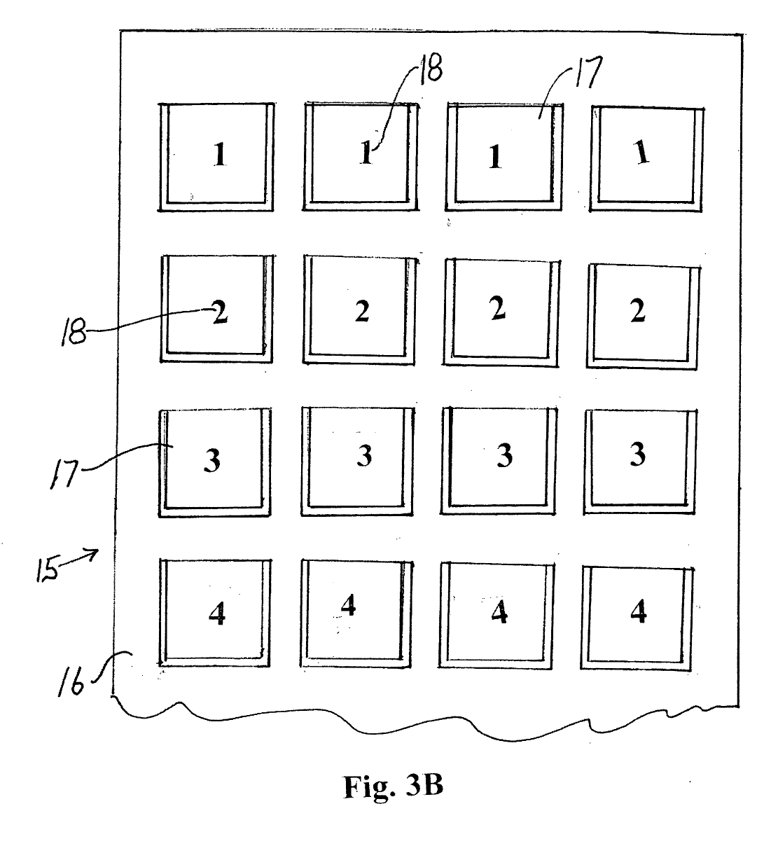

As shown in FIG. 1A, the lap pad 10 of the present invention is of the same size and construction as presently used lap pads. However, there is printed on its top face surface, or on both its top and bottom face surfaces as shown in FIG. 1B, a human readable alphanumeric character 11, or 11 and 9, respectively. Preferably each of the alphanumeric characters 9 and/or 11 are one of a series of consecutive number, for example 1,2,3 . . . 10. Each pad in a container (package) has a different number. For example, if there will be 80 lap pads estimated to be used in an operation and each container contains 40 pads, then the pads of the first container would be numbered 1-10; four groups of pads each group being numbered 1-10. The lap pads of the second container would be numbered 1 through 10, etc. Alternatively, each container may contain fewer (or more) lap pads. Each container has, in this example, four columns of pockets with each column having pockets numbered 1-10, see FIG. 3B.

The alphanumeric characters 9/11 are printed so they may be easily read by the nurses or assistants. They are 2 to 8 inches high, for example they are preferably 4 to 5 inches high. The characters 9/11 may be printed with different colors such as black and green, to distinguish one set of lap pads from the other.

FIG. 2 shows surgical sponge 12 with its printed alphanumeric character 13. Except for the size, the description of the alphanumeric characters on the lap pads are applicable to the sponges.

As shown in FIGS. 3A and B the lap pad container 15 is preferably a plastic sheet 16 having pockets 17. Each pocket 17 contains one lap pad 10. For example, a container 15 may have 40 pockets to receive 40 lap pads. Each pocket is sealed on its bottom and two opposite sides and is open at its top. The double lines 21 indicates a seal line, for example a heat seal line in a plastic container.

Each pocket is marked with the same alphanumeric character 18 as the lap pad it will contain. The pocket characters 18 are readily human readable, for example printed black characters which are 2 to 8 inches high, preferably 4 to 5 inches high.

In use the container 15 may be loaded at a factory with clean lap pads 10. Each pocket 17 will be filled with a used lap pad having the same character as the pocket's character 18. For example, the pocket marked “2” will have within it a lap pad marked “2”.

During the operation the sterile lap pads are pulled from a pack or pocket and utilized. As the operation proceeds, or near its close but before the incision is closed, the used lap pads are placed, or replaced, in the numbered pockets.

For example lap pad “6” is replaced into pocket “6”.

Near the end of the operation, all the lap pads required should have been used and placed or replaced in their marked pockets. Before the incision is closed the surgeon or nurse should visually inspect all the lap pad packages that have been used. If any pockets are empty, it would indicate that a lap pad is not accounted for, and may be still within the patient.

The present system and method improves the safety of patients. If any lap pads are missing (e.g. not in their pockets), it is immediately apparent that there is a problem. This system and method may be less costly, in terms of nurse/doctor time and yet safer than counting and re-counting surgical pads.

What is claimed is:

1. A system, in medical surgery, for accounting for surgical pads used in a surgical operation, the system including:
A. a plurality of surgical pads adapted for use in a surgical operation, each pad having a front and a back face;
B. a first set of human readable character markings, at least some of said first markings being different characters from the others of said first markings, each of said pads, on at least one said faces exhibiting at least one of said first markings;
C. a container adapted to receive used surgical pads having a plurality of pockets, each pocket having a front face and being of a suitable size and shape to receive a used pad;
D. a second set of human readable character marking which match in character the characters of the first set of markings; at least some of said second set of markings being different characters from the others of said second set of markings;
E. the front faces of at least some of said pockets exhibiting said second set of character markings; wherein each pad may be inserted into a pocket having a matching character after each pad is used.

2. A system in medical surgery as in claim 1 wherein the first and second sets of character markings are alphanumeric characters.

3. A system in medical surgery as in claim 1 wherein the first and second sets of character markings are numbers.

4. A system in medical surgery as in claim 1 wherein the first and second sets of character markings are in the range of 2 to 6 inches high.

5. A system in medical surgery as in claim 1 wherein the first character markings are printed on said front and back faces of each pad, the character on the front face of each pad being the same as the character on the back face of each pad.

6. A system in medical surgery as in claim 1 wherein the first set of character markings are printed on said pads.

7. A system in medical surgery as in claim 1 wherein the second set of character markings are printed on the pockets.

8. A system in medical surgery as in claim 1 wherein the first set of character markings are in one color and the second set of character markings are in the same color.

9. A system, in medical surgery, for accounting for surgical pads used in a surgical operation, the system including:
A. a plurality of surgical pads adapted for use in a surgical operation, each pad having a front and a back face and each pad having a radio-opaque component;
B. a first set of human readable numbers, at least some of said numbers being different numbers from the others of said numbers, each of said pads, on at least one said faces exhibiting at least one of said first set of numbers;
C. a container adapted to receive used surgical pads having a plurality of pockets, each pocket having a front face being of a suitable size and shape to receive a used pad;
D. a second set of human readable numbers which match the numbers of the first set of numbers; at least some of said second set of numbers being different numbers from the others of said second set of numbers;
E. the front faces of at least some of said pockets exhibiting said second set of numbers;
wherein pads may be inserted into pockets having matching numbers after the pads are used.

10. A system in medical surgery as in claim 9 wherein the first and second set of numbers are in the range of 2 to 6 inches high.

11. A system in medical surgery as in claim 9 wherein the first set of numbers are printed on said front and back faces of each pad, the number on the front face of each pad being the same as the number on the back face of each pad.

12. A system in medical surgery as in claim 9 wherein the first set of numbers are printed on said pads.

13. A system in medical surgery as in claim 9 wherein the second set of numbers are printed on the pockets.

14. A system in medical surgery as in claim 9 wherein the first set of numbers are in one color and the second set of numbers are in the same color.

15. A method, in medical surgery, for accounting for surgical pads used in a surgical operation, the method including:
A. providing a plurality of surgical pads adapted for use in a surgical operation, each pad having a front and a back face;
B. printing on said pads a first set of human readable character markings, at least some of said first set of markings being different characters from the others of said first markings, each of said pads, on at least one said faces exhibiting at least one of said first set of markings;
C. providing a container adapted to receive used surgical pads having a plurality of pockets, each pocket having a front face being of a suitable size and shape to receive a used pad;
D. printing on the pockets a second set of human readable character marking which match in character the characters of the first set of markings; at least some of said second set of markings being different characters from the others of said second set of markings; so that the front faces of at least some of said pockets exhibit said second set of character markings; and
E, inserting used pads into pockets having matching characters.

16. A method in medical surgery as in claim 15 wherein the first and second sets of character markings are alphanumeric characters.

17. A method in medical surgery as in claim 15 wherein the first and second sets of character markings are numbers.

19. A method in medical surgery as in claim 15 wherein the first and second character markings are in the range of 2 to 6 inches high.

20. A method in medical surgery as in claim 15 wherein the first set of character markings are printed on said front and back faces of each pad, the character on the front face of each pad being the same as the character on the back face of each pad.

21. A method in medical surgery as in claim 15 wherein the first character markings are in one color and the second character markings are in the same color.

22. A method in medical surgery as in claim 15 wherein at least some of the pads have a radio-opaque component.
